(19) United States
(12) Patent Application Publication
(10) Pub. No.: US 2017/0304112 A1
(43) Pub. Date: Oct. 26, 2017

(54) DEVICE AND METHOD FOR SPINAL COOLING

(71) Applicants: Nancy Burstein, Irvine, CA (US); Jeff Burgess, Irvine, CA (US)

(72) Inventors: Nancy Burstein, Irvine, CA (US); Jeff Burgess, Irvine, CA (US)

(21) Appl. No.: 15/498,440

(22) Filed: Apr. 26, 2017

Related U.S. Application Data
(60) Provisional application No. 62/327,565, filed on Apr. 26, 2016.

(51) Int. Cl. A61F 7/10; A41D 13/005; A61F 7/02; A61F 7/00

(52) U.S. Cl. CPC A61F 7/10; A61F 7/02; A41D 13/0056; A61F 2007/0027; A61F 2007/0233; A61F 2007/0257; A61F 2007/023

(57) ABSTRACT

A spine cooling device is configured to hold the evaporative cooling textile in contact with a wearer's skin, particularly in a location between the wearer's hips and extending toward the wearer's tailbone. The spine cooling device has a pocket to hold the evaporative cooling textile, which textile can be periodically re-wetted while the spine cooling device remains in place on the wearer. The spine cooling device facilitates direct contact between the evaporative cooling textile and the wearer's skin, and is worn beneath the wearer's clothes. However, the device includes a water resistant layer placed to prevent the evaporative cooling textile from wetting the wearer's clothes.

### CROSS-REFERENCE TO RELATED APPLICATIONS

The application claims the benefit of provisional application No. 62/327,565, which was filed on Apr. 26, 2016, the entirety of which is hereby incorporated by reference.

### BACKGROUND

The present disclosure relates to devices and methods for cooling the human body.

The human body generates heat while performing strenuous activity, such as exercise, but also generates head during daily activities. Additionally, environmental factors, such as sun exposure or environmental temperatures, contribute to body heat. The body typically regulates such heat by, for example, dilating blood vessels and/or sweating. Still, healthy people often employ external devices, such as wet towels or ice, to facilitate body cooling and increase comfort.

Multiple Sclerosis (MS) is an automimmune disorder affecting the central nervous system. MS is characterized by damage to the myelin layers of nerve fibers. Many people afflicted with MS are especially sensitive to heat, and a rise in heat may intensify MS symptoms, possibly leading to increased fatigue, leg weakness, blurred vision, decreased concentration or memory and loss of balance. Typically such intensified symptoms are temporary, and will improve as the body temperature returns to normal. It has been theorized that increased heat may reduce the damaged nerves' effectiveness in communicating signals between the brain and the body.

People with MS will often use cooling products such as vests, neck wraps, and bandanas during exercise or on warm days. Similar products are often worn by people without MS to help regulate and evacuate heat.

However, cooling products can be bulky and inconvenient, and may have limited effectiveness.

### SUMMARY

There is a need in the art for a cooling product that is convenient to use, is not bulky, and positions cooling media in a manner to maximize effectiveness.

In accordance with one embodiment, the present specification discloses a personal cooling device, comprising a pouch comprising a first liner and a second liner that are attached to one another so as to form a pocket therebetween. An opening provides access to the pocket, which pocket is configured to hold an evaporative cooling textile therewithin. A securement apparatus is configured to hold the pouch in place against a wearer's skin and beneath the wearer's clothing. The first liner is configured so that evaporating water can be communicated therethrough. The second liner comprises a water resistant layer.

One such embodiment can be combined with an evaporative cooling textile insert that is configured to be selectively held within the pocket.

Another such embodiment additionally comprises a belt configured to position an upper edge of the pouch at or adjacent the wearer's hips and aligned with the wearer's spine. In some such embodiments, the pouch depends from the belt and toward the wearer's tailbone. In additional such embodiments, the pouch is triangular and is widest at the belt. In still other embodiments, at least a portion of the belt is in contact with an outer surface of the second liner.

In additional embodiments, the first liner comprises an evaporative cooling textile.

In yet further embodiments, the first liner comprises a plurality of apertures so that at least a portion of an evaporative cooling textile insert disposed in the pocket can extend through the apertures of the first liner. In still further embodiments, the first liner comprises a mesh. Additional embodiments additionally comprise a connector configured to selectively attach the pouch to an article of clothing.

In accordance with another embodiment, the present specification describes a method of personal cooling. The method includes wetting a multilayer evaporative cooling textile insert, and arranging a pouch having a pocket so that an inner liner of the pouch is disposed adjacent a wearer's skin, the pocket defined between the inner liner and an outer liner. The pouch is secured in place. The wetted multilayer evaporative cooling textile insert is placed into the pocket so that the multilayer evaporative cooling textile insert contacts the inner liner of the pouch and conducts water from the evaporative cooling textile through the inner liner of the pouch and to the wearer's skin. An article of clothing is placed onto the wearer so that the article of clothing engages the outer liner.

In some embodiments, the outer liner does not communicate water therethrough.

In additional embodiments the inner liner comprises an evaporative cooling textile.

In yet additional embodiments, the inner liner comprises a mesh.

Still further embodiments additionally comprise applying a belt to an outer surface of the outer liner, and securing the belt so that the belt urges the pouch against the wearer's skin.

Yet further embodiments additionally comprise arranging the pouch so that it engages a back of the wearer and extends along the wearer's spine downwardly from the hips toward the tailbone.

In some such embodiments, arranging the pouch comprises adhering the pouch to a wearer's clothing.

### BRIEF DESCRIPTION OF THE DRAWINGS

FIG. 1 is a perspective view of an embodiment of an evaporative cooling textile folded to form an insert;

FIG. 2 is a top view of one embodiment of an evaporative cooling device;

FIG. 3 is a front view of a wearer wearing the cooling device of FIG. 2:

FIG. 4 is a back view of a wearer wearing the cooling device of FIG. 2;

FIG. 5A is a cross-sectional view of one embodiment of an evaporative cooling pouch taken along lines 5-5 of FIG. 2;

FIG. 5B is a cross-sectional view of another embodiment of an evaporative cooling pouch taken along lines 5-5 of FIG. 2;

FIG. 5C is a cross-sectional view of yet another embodiment of an evaporative cooling pouch taken along lines 5-5 of FIG. 2;

FIG. 6 is a top view of another embodiment of an evaporative cooling device;

FIG. 7 is a schematic view of an embodiment of an evaporative cooling pouch:

FIG. 8 is a cross-sectional view taken along lines 8-8 of FIG. 7:

FIG. 9 is a back view of a wearer wearing the cooling pouch of FIG. 7;

FIG. 10 is a schematic plan view of another embodiment of an evaporative cooling pouch;

FIG. 11 is a back view of a wearer wearing the cooling pouch of FIG. 10;

FIG. 12 is a front view of a wearer wearing another embodiment of a cooling device;

FIG. 13 is a back view of a wearer wearing the cooling device of FIG. 12;

FIG. 14A is a cross-sectional view taken along lines 14-14 of FIG. 13;

FIG. 14B is a cross-sectional view of another embodiment taken along lines 14-14 of FIG. 13;

FIG. 15 is a front view of a wearer wearing an embodiment of a cooling vest; and

FIG. 16 is a back view of a wearer wearing the cooling vest of FIG. 16.

### DESCRIPTION

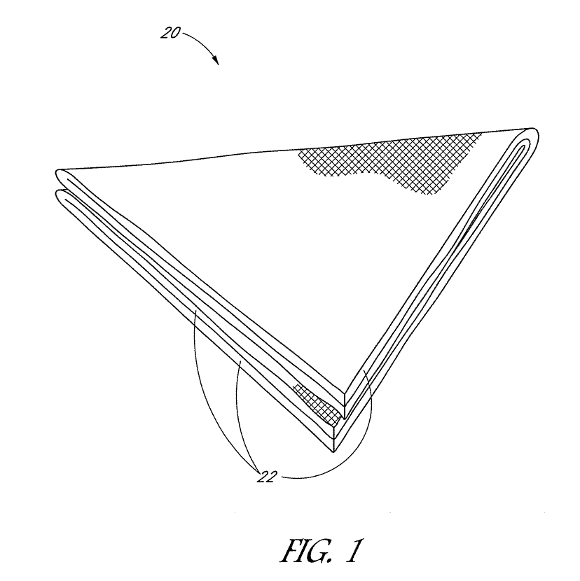

With initial reference to FIG. 1, in accordance with one embodiment, a cooling insert 20 comprises an evaporative cooling textile that has been formed into a desired shape, which is a triangle in the illustrated embodiment. In this embodiment, the evaporative cooling textile is formed of a fabric that is specifically adapted to regulate, or slow, the rate of evaporation of water entrained in the textile. As such, when wetted and placed into contact with a wearer's skin, the evaporative cooling textile facilitates evaporative cooling of the site of contact. One preferred evaporative cooling textile is currently sold under the name EnduraCool, and can be obtained in various configurations, such as a rectangular towel and/or a bandana. This textile can be obtained in versions made from 90% polyester and 10% nylon, or 92% polyester and 8% spandex. It is to be understood that other evaporative cooling textiles can be obtained from other sources, and may be appropriately used with embodiments discussed herein.

In use, the evaporative cooling textile is wetted, wrung out and, in some embodiments, snapped in the air in order to activate its cooling properties. In a preferred embodiment, the activated evaporative cooling textile is folded over itself multiple times into a desired shape to form an insert 20. In the illustrated embodiment, the activated textile is folded in a manner similar to a US flag in order to produce a triangular insert 20. As a result of folding, the insert 20 includes several layers 22 of evaporative textile.

In a preferred embodiment, the insert 20 is folded for use, and can be unfolded for cleaning, re-wetting, wringing out, and the like. In additional embodiments, the insert 20 can be formed of several layers 22 formed into the desired shape, but sewn or otherwise bonded together so that the desired shape is retained.

As will be discussed in more detail below, preferably, the cooling insert 20 is placed adjacent the wearer's skin in the area of the spine at and below the hips, concentrating cooling in this area. Applicants have found that cooling this particular area of the body is particularly effective in providing heat relief to and asked patients individuals suffering hot flashes due to menopause or other causes, and individuals performing strenuous activities and/or working in warm environments.

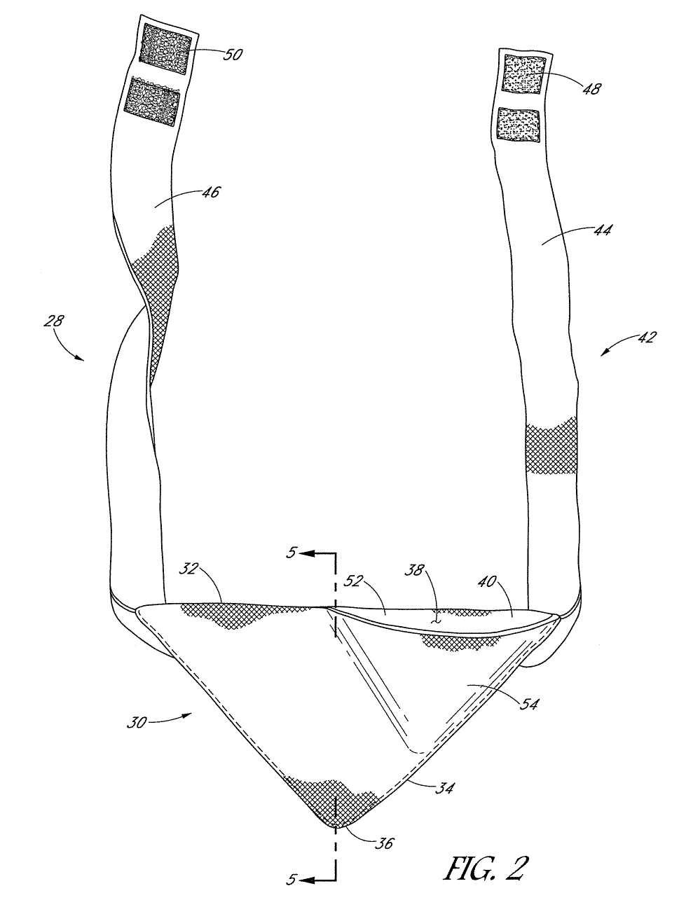

With reference next to FIGS. 2-5, a removable cooling device 28 comprises a generally triangular triangular-shaped pouch 30 having a top edge 32 and a bottom edge 34. In the illustrated embodiment, the top edge 32 is generally straight and the bottom edge before comprises two generally straight portions defining the triangular shape. A peak 36 of the pouch 30 is the point farthest from the top edge 32. As such, the width of the pouch 30 is greatest along the top edge 32 and decreases moving toward the peak 36. An opening 38 is defined along the top edge 32, and a pocket 40 is accessible through the opening 38. Preferably, the pocket 40 is defined between an inner liner 52 and an outer liner 54 of the pouch 30.

The pocket 40 preferably is sized and shaped to generally complement the size and shape of the insert 20 so that the insert 20 can be readily inserted into the pocket 40 and held within the pouch 30.

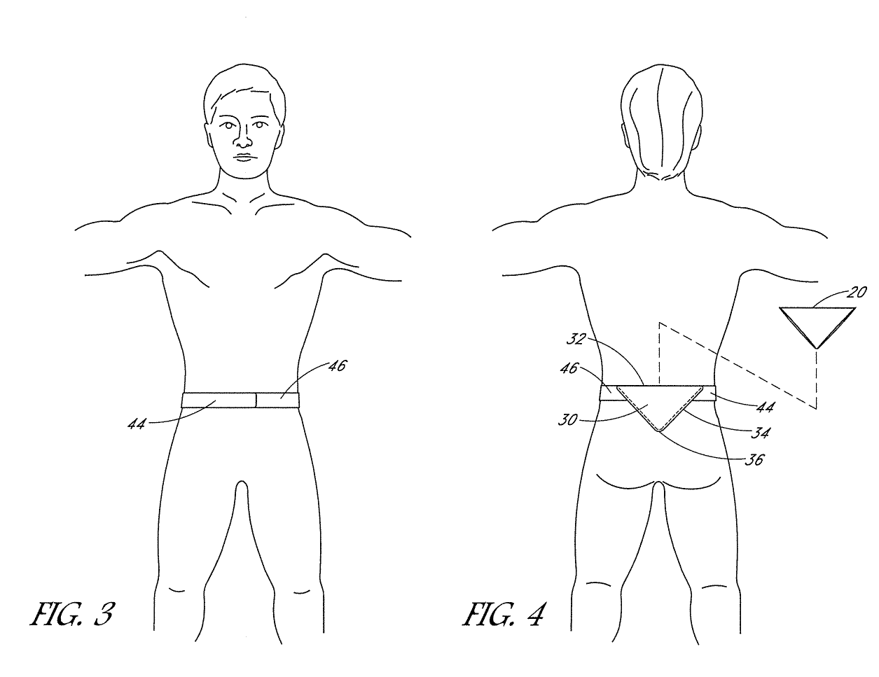

A belt 42 is attached to the pouch 30 and configured to secure the removable cooling device 28 to a wearer, as demonstrated in FIGS. 3 and 4. The illustrated belt 42 comprises a first strap 44 attached to one side of the pouch 30 and a second strap 46 attached to an opposing side of the pouch 30. A first connector 48 on the first strap 44 is configured to releasably engage a second connector 50 on the second strap 46. In a preferred embodiment, the connectors 48, 50 are formed from a hook and loop type of fastener. It is to be understood, however, that any of several connecting structures, including snaps buttons, buckles or the like can be employed.

As best shown in FIG. 4, in a preferred embodiment, the removable cooling device 28 is worn by the wearer by securing the belt 42 around their waist so that the top edge 32 of the pouch 30 is positioned at or adjacent the wearer's hips. The pouch 30 depends downwardly from this position so that it extends toward the wearer's tailbone. As such, a lumbosacral portion of the wearer's spine, generally below the waist, is covered by the pouch 30.

Preferably, the pouch 30 is worn under a wearer's clothing so that it is in contact with the wearer's skin. More specifically, the inner liner 52 is in direct contact with the wearer's skin. As such, when the insert 20 is placed within the pocket 40, it will be in a position to effectively cool the wearer's skin.

In the illustrated embodiment, the straps 44, 46 are attached to edges of the pouch 30 at and adjacent the top edge 32. In additional embodiments, the straps 44, 46 may be attached to the outer liner 54 so that when the device 28 is worn as shown in FIGS. 3 and 4, the straps 44, 46 will urge the outer liner 54, and thus the insert 20 within the pocket 40, toward and into contact with the wearer's skin.

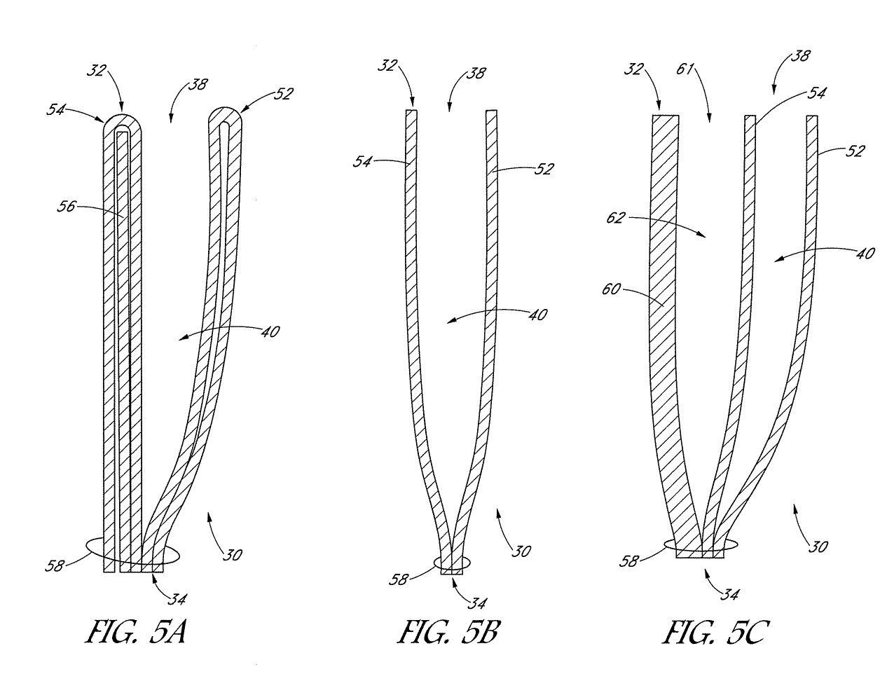

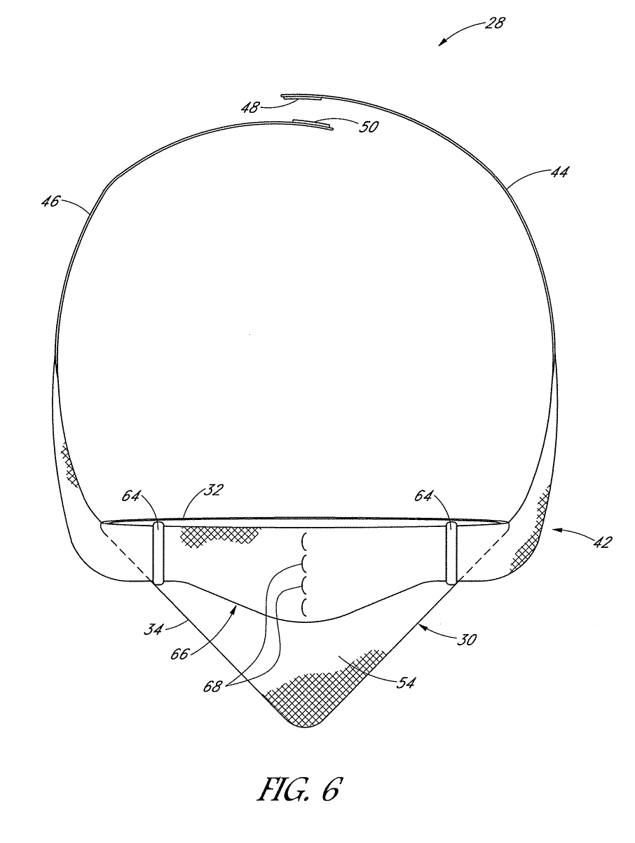

With particular reference to FIG. 5A, a cross-section of one embodiment of a pouch 30 is shown schematically. In the illustrated embodiment, the pouch 30 comprises a first or inner liner 52 and a second or outer liner 54. The inner and outer liners 52, 54 preferably are joined along the bottom edge 34, such as by stitching 58, thus leaving the opening 38 adjacent the top edge 32. The pocket 40 is defined between the inner and outer liners 52, 54. As noted above, preferably a cooling insert 20 can be inserted through the opening 38 so as to be held securely in the pocket 40.

In the illustrated embodiment, the inner liner 52 is formed of flat textile that has been folded over itself, resulting in two layers. Similarly, the outer liner 54 is formed of a flat textile that has been folded over itself, resulting in two layers. However the outer liner 54 encloses an enclosed layer 56 between the folded layers. In a preferred embodiment, the inner liner 52 and folded textile portion of the outer liner 54 are made of the same evaporative cooling textile as the insert 20. Thus, in use a wearer not only wets the insert 20, but also wets at least the inner liner 52 of the pouch 30 (and perhaps one layer of the outer liner 54) in order to maximize and prolong the cooling effect. It has been determined that having a plurality, even several, layers of evaporative cooling textile prolongs the duration of the cooling effect, and in some embodiments the liners 52, 54 can provide additional layers of evaporative cooling textile.

With continued reference to FIG. 5A, in the illustrated embodiment, the enclosed layer 56 comprises a water resistant layer. The water resistant layer 56 can take many forms. For example, it can comprise a layer of plastic, water resistant textile such as Gore-Tex®, or natural or artificial fiber-based fabric that has been treated with a water resistant substance or laminated to a water resistant substance. Preferably, the wearer wears the cooling device 28 underneath his or her clothing so that the inner liner 52 is in direct contact with the wearer's skin. The wearer's clothing thus is in contact with the outer liner 54. The water resistant enclosed layer 56 blocks water from the pouch 30 and insert 20 from wetting the wearer's clothes. However, preferably there is no layer blocking or inhibiting evaporative cooling textile from directly contacting the wearer's skin.

In some embodiments, the entire removable cooling device 28 can be substantially formed of evaporative cooling textile, such as the evaporative cooling textile used for the insert 20. In additional embodiments, at least portions of the device 28 can be made of different materials. For example in one embodiment the straps 44, 46 are formed of an elastic and/or mesh material that may or may not have evaporative cooling properties. In some embodiments, the straps 44, 46 are formed of material that substantially does not absorb water so that if the device 28 is wetted, the straps 44, 46 substantially will not remain wet and presumably will not in turn substantially wet the wearer's clothes.

With reference next to FIG. 5B, a schematic cross-sectional view of another embodiment of the pouch 30 is shown. In the illustrated embodiment, the first or inner liner 52 is formed of a single layer, which single layer may in some embodiments comprising evaporative cooling textile. In additional embodiments, the inner liner 82 comprises a mesh. As such, when the insert 20 is placed within the pocket 40, the insert 20 can directly access and contact the wearer's skin through the mesh inner liner 52 without substantial inhibition of such contact. In the illustrated embodiment, the second or outer liner 54 comprises a layer of water resistant textile. As such, the pouch 30 of FIG. 5B can be made very thin and unobtrusive, yet facilitate direct contact of the cooling insert 20 with the wearer's skin while simultaneously protecting the wearer's clothing from being wetted by the insert 20. In such an embodiment, there is no substantial benefit from wetting the pouch 30, as substantially all of the evaporating cooling effect will be provided by the insert 20.

With reference next to FIG. 5C, a schematic cross-sectional view of yet another embodiment of the pouch 30 is shown. In the illustrated embodiment, the first and second liners 52, 54 are bonded to a third liner 60. As such, opening 38 is formed between the first and second liners 52, 54 and provides access to pocket 40, and an opening 61 is formed between the second and third liners 54, 60 and provides access to a second pocket 62 is formed between the second and third liners 54, 60.

In the illustrated embodiment, an evaporative cooling insert 20 can be placed in the first pocket 40, and a secondary cooling device can be placed in the second pocket 62. Such secondary cooling devices can have various structures and configurations. For example, in one embodiment, a secondary cooling device can comprise an ice pack or other cold pack, which can be configured to enhance the effect of the evaporative cooling insert 20 and the adjacent first pocket 40. In such embodiments, it may be desired for the third liner 60 to be formed of an insulating material so that the third liner 60 operates as an insulated layer, keeping the cold pack cool. Correspondingly, the second liner 54 can be a relatively thin layer figured to facilitate cold transfer from the second pocket 62 to the first pocket 40.

In additional embodiments, the secondary cooling device can comprise a sponge having a higher water content than the cooling insert 20. As water evaporates from the cooling insert 20 while cooling the wearer, water from the sponge may replenish the evaporated water, prolonging and/or enhancing the cooling effect on the wearer. In still another embodiment, the secondary cooling device can be another insert evaporative cooling insert, and the second liner 54 can comprise a mesh courage contact between the inserts in the first pocket 40 and second pocket 62 or, in another embodiment, the second liner can also comprise an evaporative cooling textile. Devices, such as an airflow generation device comprising one or more small fans, can also be placed in the second pocket 62.

With reference next to FIG. 6, in another embodiment of a removable cooling device 28, the belt 42 is configured to circle around and contact the outer surface of the outer liner 54. Preferably, a center portion of the belt is connected to a central portion of the outer liner 54 such as by stitches 68. Preferably, the belt 42 is formed of a flexible or elastic layer that stretches when worn by the wearer. As such, when worn and tightened, the belt 42 urges the pouch 30, and thus the insert 20, and to contact with the wearer's skin. In the illustrated embodiment, the straps 44, 46 of the belt 42 are threaded through loops 64 formed on the outer liner 54 of the pouch 30. The loops 64 enable movement of the straps 44, 46 when the straps are stretched, but maintain them in their position on the outer surface of the outer liner 54. In the illustrated embodiment, an expanded portion 66 of the belt 42 is engaged against the outer liner 54 of the pouch 30. Since the belt 42 is wider in the expanded portion 66, it distributes forces across the outer liner 54 and urges a greater portion of the pouch 30 against the wearer's skin.

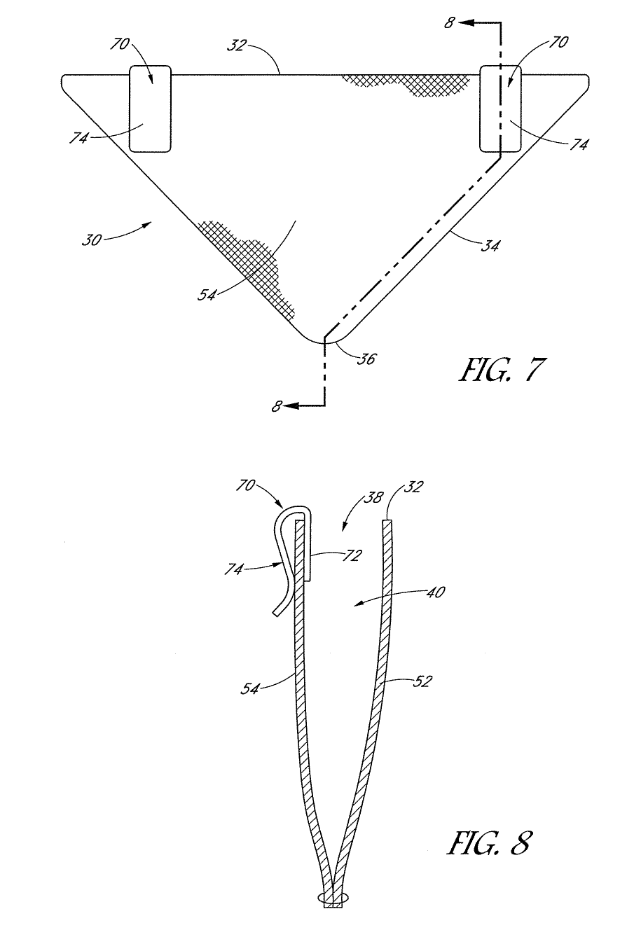

With next referenced to FIGS. 7-9, in another embodiment, a pouch 30 may not be supported by a belt. Instead, a pair of connectors 70 are attached to the outer lining 54, at or adjacent the upper edge 32. The connectors 70 are configured to engage and attach to the wearer's clothing in order to maintain the pouch 30 in its appropriate place against the wearer's skin at and below wearer's hips. In the illustrated embodiment, each connector 70 comprises a base 72 that is attached to an inner surface of the outer lining 54. Such attachment can be accomplished by way of an adhesive, stitching, or other methods or structure for attachment to textiles. A tab 74 of the connector 70 extends from the base 72 over the upper edge 32 and overlaps an outer surface of the outer lining 54.

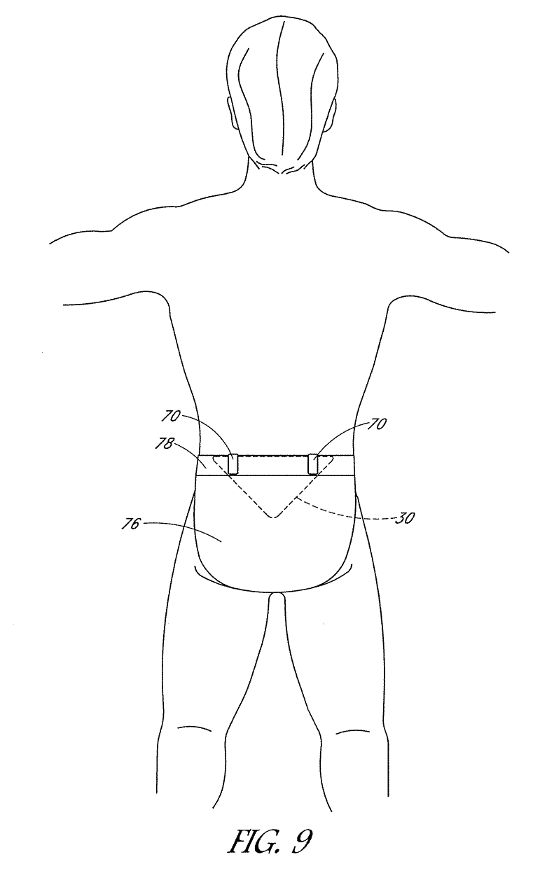

With continued reference to FIGS. 7-9, the illustrated tabs 74 are constructed so that, as best shown in FIG. 9, the connectors can be clipped over the waistband 78 of the wearer's underwear 76 so that the underwear 76 supports the pouch 30 in the proper position and holds the pouch against the wearer's skin.

It is to be understood that, in other embodiments, other methods and structures for attaching a pouch 32 a wearer's clothing can be used for the connectors 70. For example, some connectors may be considered so as to engage buttons that may be provided on the wearer's clothing. Other connectors may include an adhesive layer configured to engage and stick to a wearer's clothing. Further connectors can include pins, hook-and-loop fasteners, or the like to releasably attach the pouch to the wearer's clothing.

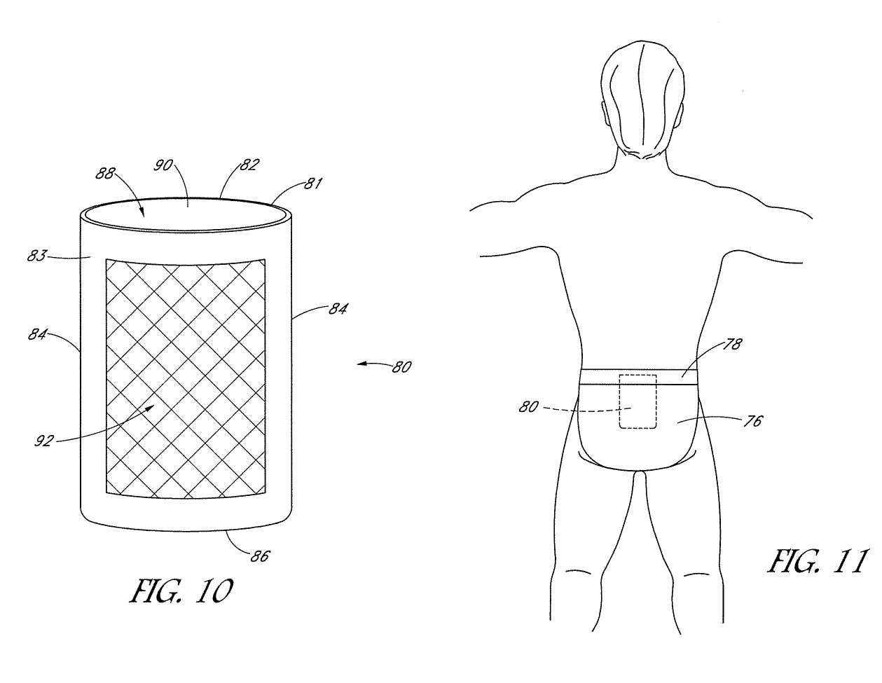

With reference next to FIGS. 10 and 11, in yet another embodiment, a pouch 80 may be configured with a rectangular shape that is configured to contact the wearer's skin and extend along he wearer's spine at least from the area at and adjacent the hips toward the tailbone. The illustrated pouch 80 comprises an inner lining 81 and an outer lining 83 that are joined together to form side edges 84 and a bottom edge 86, but which are not joined together along their top edges 82. As such, an opening 88 is formed between the inner and outer liners 81, 83 at the top edges 82. A pocket 90 is defined between the inner liner 81 and the outer liner 83, and is accessible through the opening 88.

In the illustrated embodiment, adhesive layer 92 is formed on the outer surface of the outer lining 83. Most preferably, a release liner (not shown) is provided to cover and protect the adhesive layer 92. In use, the release liner can be removed, exposing the adhesive 92, which can then be placed into contact with an inner surface of the wearer's undergarments 76 so as to be positioned adjacent the lumbosacral portion of the wearer's spine generally at and below the hips, as shown in FIG. 11. An evaporative cooling insert can be folded to a rectangular shape that will fit into the pocket 90.

In some embodiments, the pouch 80 is formed of a low-cost material so that the pouch 80 can be discarded after use. For example, the inner lining 81 can be formed of a low-cost natural or synthetic mesh, while the outer lining 83 can be formed of any of several disposable fabrics such as disposable medical fabrics that preferably provide water resistance. Examples of suitable fabrics include, for example, polyethylene coated polypropylene, non-woven spunlace, or the like.

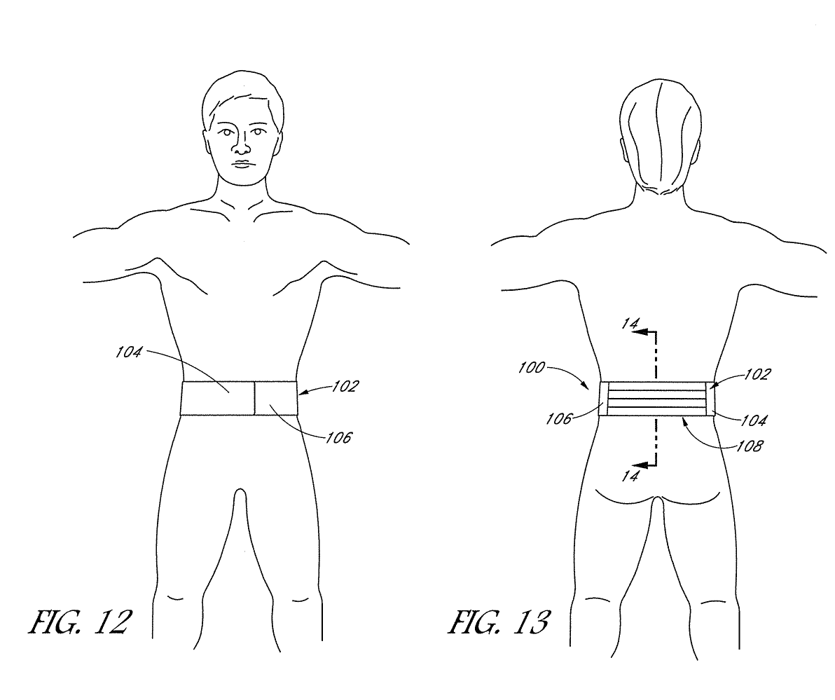

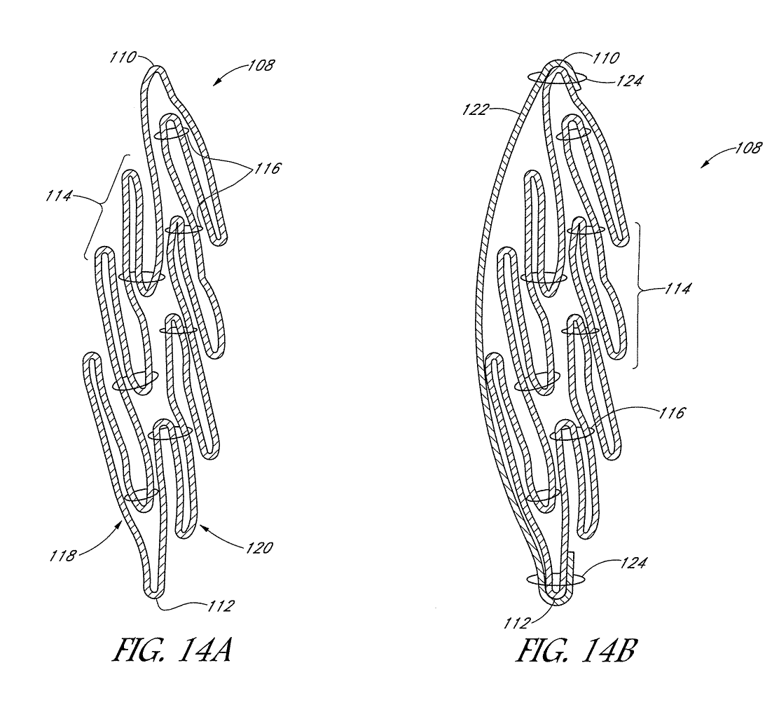

With reference next to FIGS. 12-14, an embodiment of a waist cooling device 100 is presented. The illustrated waist cooling device 100 comprises a waist belt 102 configured to fit about the wearer's waist, generally above the hips, as shown in FIGS. 12 and 13. The belt 102 preferably comprises a first strap 104 and a second strap 106 that are configured to be secured about the wearer's waist to hold the cooler 100 in place. A concentrated zone 108 of the cooler 100 is configured to be arranged on the wearer's back. As best shown in FIG. 14A, the concentrated zone 108 has an upper edge 110 and a lower edge 112. A plurality of overlapping portions 114 are created by overlapping the textile and attaching the overlapping portions 114 to one another using various media, which can include stitches 116. In the illustrated embodiment, the concentrated zone 108 is made of evaporative cooling textile. As such, overlapping portions 114 provide multiple layers of such evaporative cooling textile.

In some embodiments, the entire waist cooling device 100 is formed of evaporative cooling fabric, including the belt 102. In other embodiments, the belt 102 is formed of a different textile such as an elastic and/or mesh material, and only the concentrated zone 108 is formed of evaporative cooling textile.

With reference next to FIG. 14B, in another embodiment, the concentrated zone 108 can comprise an outer liner 122 covering an entire side of the zone 108 from the upper edge 110 to the lower edge 112 and being bonded thereto, such as via stitches 124. Preferably, the overlapping portions 114 provide multiple layers of evaporative cooling textile to, as discussed above, prolong and improve the cooling effect. The illustrated evaporative cooling zone provides a first row 118 of overlapping portions 114 and a second row 120 opposite side of the zone one. The outer liner 122 preferably is formed of a water resistant material. Preferably the waist cooling device 100 is configured so that the outer liner 122 faces the wearer's clothing, and a row of the overlapping portions 114 of the evaporative textile are in contact with the wearer's skin.

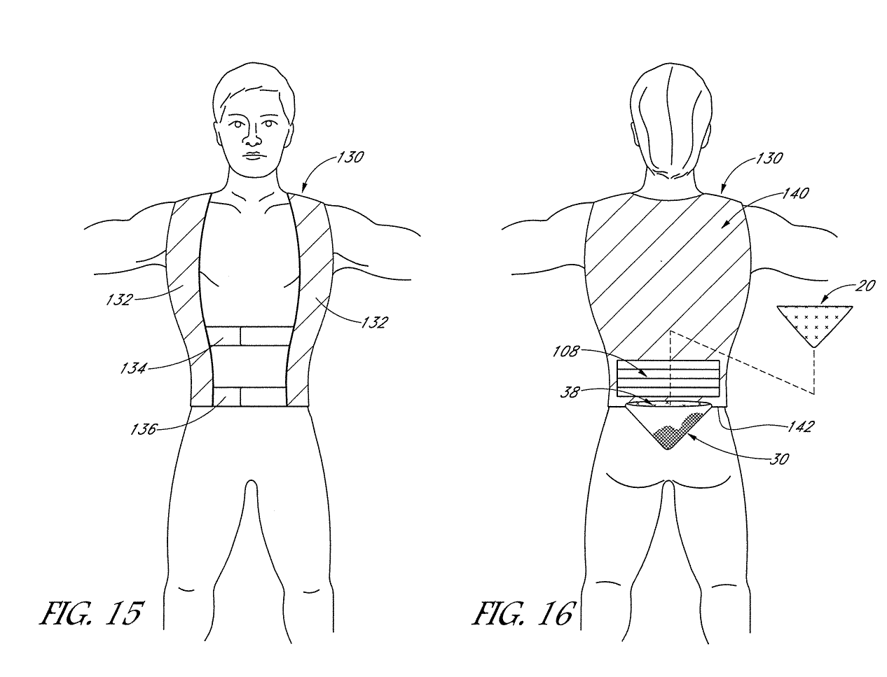

With reference next to FIGS. 15 and 16, in accordance with yet another embodiment, a cooling vest 130 comprises front portions 132 that are held together by a first strap pair 134 and second strap pair 136. The front portions 132 are connected to a back portion 140. Preferably the strap pairs 134, 136 pull the front portions 132 together, promoting a snug fit of the Cooling vest 130. In a preferred embodiment, the front portions 132 and back portion 140 are formed of an evaporative cooling textile. Preferably, the vest 130 is sized and configured so that a bottom edge 142 of the vest 130 is at or slightly above the wearer's hips when worn.

With continued reference to FIG. 16, in a preferred embodiment, a pouch 30 is attached to the back 140 of the vest 130 and configured so that the pouch 30 hangs downwardly beyond the lower edge 142 of the vest. As such, when worn by the wearer, the vest 130 positions the pouch 30 adjacent the wearer's skin at and below the hips. Preferably, the illustrated pouch 30 has structure and operation similar to embodiments of the pouch 30 discussed above, and end evaporative cooling insert 20 can selectively be inserted through the opening 38 and into the pouch 30 to provide a cooling effect to the wearer.

Continuing with reference to FIG. 16, in the illustrated embodiment, the back portion 140 includes a concentrated zone 108 positioned at the wearer's waste adjacent but above the hips. Preferably the concentrated zone 108 is configured similarly to embodiments of the concentrated zone 108 discussed above in connection with FIGS. 12-14.

It is to be understood that additional embodiments may employ variations and hybrids of the structures disclosed herein. For example, although the vest 130 depicted in FIG. 16 employs a generally triangular pouch 30, pouches having other configurations, such as a rectangular configuration as used in FIG. 10, may be employed. Also in some embodiments, inner liners of pouches may employ overlapping structure such as is used in connection with the illustrated concentrated zone 108. Additionally, some cooling devices may employ combinations of the structures disclosed herein. For example, a waist cooling device such as the device 100 discussed in connection with FIGS. 12-13 may also include a pouch 30 depending therefrom as does the vest 130 of FIG. 16. Additionally, adhesive layers can be employed in connection with pouches of various configurations, whether formed of disposable materials or not.

The embodiments discussed above have disclosed structures with substantial specificity. This has provided a good context for disclosing and discussing inventive subject matter. However, it is to be understood that other embodiments may employ different specific structural shapes and interactions.

Although inventive subject matter has been disclosed in the context of certain preferred or illustrated embodiments and examples, it will be understood by those skilled in the art that the inventive subject matter extends beyond the specifically disclosed embodiments to other alternative embodiments and/or uses of the invention and obvious modifications and equivalents thereof. In addition, while a number of variations of the disclosed embodiments have been shown and described in detail, other modifications, which are within the scope of the inventive subject matter, will be readily apparent to those of skill in the art based upon this disclosure. It is also contemplated that various combinations or subcombinations of the specific features and aspects of is configured to be worn by a user as a front portions the disclosed embodiments may be made and still fall within the scope of the inventive subject matter. Accordingly, it should be understood that various features and aspects of the disclosed embodiments can be combined with or substituted for one another in order to form varying modes of the disclosed inventive subject matter. Thus, it is intended that the scope of the inventive subject matter herein disclosed should not be limited by the particular disclosed embodiments described above, but should be determined only by a fair reading of the claims that follow.

What is claimed is:

1. A personal cooling device, comprising:
a pouch comprising a first liner and a second liner that are attached to one another so as to form a pocket therebetween, an opening providing access to the pocket, the pocket configured to hold an evaporative cooling textile therewithin; and
a securement apparatus configured to hold the pouch in place against a wearer's skin and beneath the wearer's clothing;
wherein the first liner is configured so that evaporating water can be communicated therethrough; and
wherein the second liner comprises a water resistant layer.

2. A personal cooling device as in claim 1, in combination with an evaporative cooling textile insert configured to be selectively held within the pocket.

3. A personal cooling device as in claim 1 additionally comprising a belt configured to position an upper edge of the pouch at or adjacent the wearer's hips and aligned with the wearer's spine.

4. A personal cooling device as in claim 3, wherein the pouch depends from the belt and toward the wearer's tailbone.

5. A personal cooling device as in claim 4, wherein the pouch is triangular, and is widest at the belt.

6. A personal cooling device as in claim 4, wherein at least a portion of the belt is in contact with an outer surface of the second liner.

7. A personal cooling device as in claim 1, wherein the first liner comprises an evaporative cooling textile.

8. A personal cooling device as in claim 1, wherein the first liner comprises a plurality of apertures so that at least a portion of an evaporative cooling textile insert disposed in the pocket can extend through the apertures of the first liner.

9. A personal cooling device as in claim 8, wherein the first liner comprises a mesh.

10. A personal cooling device as in claim 9, additionally comprising a connector configured to selectively attach the pouch to an article of clothing.

11. A method of personal cooling, comprising:
wetting a multilayer evaporative cooling textile insert;
arranging a pouch having a pocket so that an inner liner of the pouch is disposed adjacent a wearer's skin, the pocket defined between the inner liner and an outer liner;
securing the pouch in place;
placing the wetted multilayer evaporative cooling textile insert into the pocket so that the multilayer evaporative cooling textile insert contacts the inner liner of the pouch and conducts water from the evaporative cooling textile through the inner liner of the pouch and to the wearer's skin; and
placing an article of clothing onto the wearer so that the article of clothing engages the outer liner.

12. A method as in claim 11, wherein the outer liner does not communicate water therethrough.

13. A method as in claim 11, wherein the inner liner comprises an evaporative cooling textile.

14. A method as in claim 11, wherein the inner liner comprises a mesh.

15. A method as in claim 11 additionally comprising applying a belt to an outer surface of the outer liner, and securing the belt so that the belt urges the pouch against the wearer's skin.

16. A method as in claim 11 additionally comprising arranging the pouch so that it engages a back of the wearer and extends along the wearer's spine downwardly from the hips toward the tailbone.

17. A method as in claim 15, wherein arranging the pouch comprises adhering the pouch to a wearer's clothing.
